(19) United States
(12) Patent Application Publication
(10) Pub. No.: US 2021/0292814 A1
(43) Pub. Date: Sep. 23, 2021

(54) METHOD FOR SCREENING APTAMER BY USING MICROARRAY MICROFLUIDIC CHIP

(71) Applicant: NANJING UNIVERSITY, Nanjing (CN)

(72) Inventors: Danke XU, Nanjing (CN); Hui LI, Nanjing (CN); Xiaohui LIU, Nanjing (CN); Zhu CHEN, Nanjing (CN)

(21) Appl. No.: 17/336,112

(22) Filed: Jun. 1, 2021

Related U.S. Application Data
(63) Continuation-in-part of application No. 16/340,082, filed on Apr. 5, 2019.

(51) Int. Cl. C12Q 1/686; C12Q 1/6853

(52) U.S. Cl. CPC C12Q 1/686; C12Q 1/6853

(57) ABSTRACT

A method for screening aptamers by using a microarray microfluidic chip. The screening chip integrates microarray and microfluidic technology to integrate the positive and negative screening process on a microfluidic chip, and obtains aptamers with high affinity after 7 rounds of screening. At the same time, the present invention also discloses specific steps for screening of lactoferrin aptamers, including detailed processes such as chip preparation, positive and negative screening processes, and PCR amplification. The aptamers screened by the method have good specificity and affinity to the target protein. The aptamers are easier to be obtained than the antibody, and can be synthesized rapidly in large quantities in vitro. The preparation method is simpler and faster, so aptamers are expected to be a useful complement to antibody technology in many areas.

This application is a continuation-in-part application of U.S. Ser. No. 16/340,082 that is the U.S. national phase of International Application No. PCT/CN2017/073024 filed on 7 Feb. 2017 which designated the U.S. and claims priority to Chinese Application No. CN201610894672.0 filed on 13 Oct. 2016, the entire contents of each of which are hereby incorporated by reference.

### TECHNICAL FIELD

The invention belongs to the technical field of biological detection, and particularly relates to a method for screening aptamers by using a microarray microfluidic chip.

### BACKGROUND TECHNIQUE

The aptamer has similar properties to the antibody, but it also exhibits superior characteristics to the antibody in many respects. (1) Aptamers are more specific with higher affinity for the target molecule than the antibody. (2) Aptamers are easier to obtain than antibodies (synthetically, independent of animals and cells), and can be synthesized quickly in vitro with large quantities, making preparation methods simpler and faster. (3) Different types of target molecules can be screened, including bio-toxic and semi-antigenic molecules, broadening the scope of application. (4) Aptamers are non-immunogenic and can be used repeatedly in the body. (5) Aptamers' stability is better than antibodies and conducive to storage. With the advancement of aptamer technology, aptamers will likely be a useful complement to antibody technology in many fields. Based on various advantages of aptamers, they have shown broad application prospects in analysis, clinical, environment, molecular recognition and drug screening.

At present, nanomaterial-based aptamer screening technology can eliminate the immobilization of proteins on the surface of solid phase substrates, which can effectively reduce the number of screening rounds and speed up the screening process. At the same time, the method based on microfluidic technology to improve the screening effect has been extensively studied. These methods include: capillary electrophoresis microfluidic aptamer screening, sol-gel microfluidic aptamer screening, surface plasmon resonance (SPR) microfluidic aptamer screening, magnetic bead microfluidic screening, and microfluidic screening techniques based on microspheres of various materials. In order to improve the efficiency of PCR amplification, scientists use agarose droplet PCR amplification technology and poly-emulsion PCR amplification (ePCR) technology, which can directly select positive clones for sequencing, avoiding the cumbersome process of cloning and sequencing in the traditional aptamer screening process. The above methods are intended to make the screening of aptamers more time-saving, automated, efficient and less expensive. Our job is to develop a new aptamer screening technique that is more stable, the screening process is repeatable, and the screening steps are quantifiable. The method optimizes the process of aptamer screening to make the process more simple and standardized.

### SUMMARY OF THE INVENTION

The technical problem to be solved by the present invention is to provide a novel microarray microfluidic chip for applying to an aptamer screening method.

In order to solve the above technical problems, the technical solution adopted by the present invention is as follows:

A method for screening aptamers by using a microarray microfluidic chip, the microarray microfluidic chip is prepared as follows:
The PDMS pre-polymer and the curing agent are mixed at a mass ratio of 5:1 to 100:1. After being uniformly mixed, they are placed in a vacuum desiccator which is connected to a circulating water vacuum pump. The mixture are vacuumed for 30 min to remove bubbles, and then are poured on the microfluidic channel template. After curing, PDMS microfluidic channel is obtained. Lactoferrin and negative protein microarray with concentration of 0.1-10 mg/mL are prepared on the glass substrate. Then, the glass substrate and PDMS microfluidic channel are simultaneously plasma-treated, and closely adhered to obtain a microarray microfluidic chip.
The method for screening an aptamer using a microarray microfluidic chip according to claim 1, comprising the steps of:
(1) Screening preparation: The microarray microfluidic chip is incubated at a constant temperature for 1-12 h at 25-40° C. 5-20 mg/ml of BSA and 0.01-0.5 mM of random sequence short-chain ssDNA (40 nt) are added at 25-40° C. for 1-3 h, then the positive and negative channel on chip are cleaned by 150 μL of 1×PBST solution;
(2) The first round of screening: 0.1-10 nmol, 125 μL of the original library are heated at 95° C. for 3-15 min, and immediately frozen on ice for 10 s-5 min. The library are transferred at a flow rate of 1-5 μL/min by syringe pump into the positive channel, reacting at 25-40° C. for 30-120 min. then 150 μL of 1×PBS buffer was injected at a flow rate of 5-30 μL/min to remove the unbound chain in the positive channel. The PDMS layer is torn off and microarray scanner is used to scan the chip. Finally, the lactoferrin-bound ssDNA is eluted by heating with DPEC water at 95° C. for 3-10 min, and the resulting solution was dried to a volume of 25-250 μL with high purity nitrogen at 40-60° C.
(3) PCR amplification process: the solution obtained in step (3) is divided into 3 to 10 parts of the same solution with a volume of 23 μL. Each solution is added with 25 μL of 2×Taq polymerase, 1 μL of 20 μM TAMRA labeled forward primer and 1 μL of 20 μM biotinylated backward primer for PCR amplification.

The product obtained in this step was diluted 5 to 40 times as a template and then amplified again. 5 μL of diluted PCR product with 1 μL of 2×SYBR Premix Ex Taq™ enzyme, 1 μL of 20 μM TAMRA-labeled forward primer, 1 μL of 20 μM biotinylated backward primer and 18 μL of ultrapure water were mixed uniformly for PCR amplification. 10 μL of sample was taken for fluorescence in microplate by BioTek. The round number with highest fluorescence signal was selected to amplify the remaining products.

(4) Separation and purification: The obtained PCR products in step (3) were mixed with 800 μL-5 mL of Promega magnetic beads, the supernatant are removed after rapid shaking for 0.5-2 h. 25 μL of 0.01-0.1 M NaOH solution are added, the double stands are dissociated from the magnetic beads after vortex oscillation for 3-15 min. Then 12.5 μL of 0.1M HCl, 25 μL of DPEC water and 62.5 μL of 2×PBSM buffer solution are added to the supernatant. Neutral solution as a secondary library for the next round of screening with concentration of 40 pmol-100 pmol.
(5) The second round of screening: The library above was sequentially transported into the negative channel at 1-5 μL/min at 25-40° C., and then into the positive channel. Then 150 μL of 1×PBS buffer solution at 5-30 μL/min flow rate to remove unreacted ssDNA sequence in the positive channel. The PDMS layer is gently torn off and Luxscan-10K/A microarray scanner is used to scan the chip. Finally, DPEC water at 95° C. is used to elute the ssDNA sequence bound to the lactoferrin on the chip by heating for 3-10 min, and the obtained solution was dried to 20-250 μL at 60° C. for PCR amplification.
(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;
(8) repeating steps (5), (6) and (7) for 2^{nd}-6^{th} round screens, and repeating steps (5) and (6) for 7^{th} round screen;
(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.
The above screening process is repeated and the samples are randomly selected for sequencing. All sequences with a repeated sequence greater than 2 are obtained, and the obtained repeated sequences are performed by IDT software. IDT software automatically generates possible secondary structure and ΔG according to the sequence. The end of the sequence will be secondly trimmed to find out a more efficient sequence and a more stable secondary structure (minimum ΔG value) to obtain the optimal aptamer.
In the step (2), the library has 40 random bases in the middle, the nucleotide sequence at the 5′ end of the random base is as shown in SEQ ID NO. 1, and the nucleotide sequence at the 3′ end of the random base is as shown in SEQ ID NO.2.
In the step (3), the forward primer has a nucleotide sequence as shown in SEQ ID NO. 3.

The nucleotide sequence of the backward primer is shown in SEQ ID NO.4.

An aptamer for detecting lactoferrin, wherein the aptamer has a nucleotide sequence as shown in SEQ ID NO. 5-65.

The use of the above aptamers for detecting lactoferrin content in the detection of lactoferrin is within the scope of the present invention.

The principle of the invention: the aptamer is produced by a systematic evolution of ligands by exponential enrichment (SELEX), which can be used to screen specific nucleic acid sequence from random single-stranded nucleic acid sequence libraries. The nucleic acid ligand (aptamer) has highly affinity with the target substance. The invention is based on microarray technology with the large-scale, high-throughput, small volume and microfluidic technology with automatic fluid transport, enhanced molecular reaction efficiency and closed reaction chamber avoiding external pollution. The negative and positive screening process of the aptamer screening are combined on a microfluidic chip, and the aptamer screening can be completed quickly and efficiently. By screening the fluorescence intensity changes after the ssDNA sequence is specifically captured on the positive channel, the evolution of the secondary library with negative screening and positive screening in each screening process can be directly and intuitively monitored. After the method was screened for 7 rounds, aptamer with high affinity are obtained.

The method of screening the aptamers by this microarray microfluidic chip is designed for the first time. Protein microarray production methods, screening libraries and protein array process monitoring are used universally. In addition, PCR process, PCR product purification, secondary library regeneration process are used universally. Therefore, the method of screening aptamers using microarray microfluidic chips has a good general purpose and important research significance.

### BENEFICIAL EFFECTS

The invention discloses a method for screening an aptamer by using a microarray microfluidic chip. The screening chip integrates microarray and microfluidic technology to integrate the positive and negative screening process on a microfluidic chip, and obtains aptamer with high affinity after 7 rounds of screening. At the same time, the present invention also discloses specific steps for screening of lactoferrin aptamers, including detailed processes such as chip preparation, positive and negative screening processes, and PCR amplification. The aptamer screened by the method has good specificity and affinity to the target protein. The aptamer is easier to be obtained than the antibody, and can be synthesized rapidly in large quantities in vitro. The preparation method is simpler and faster, so aptamers are expected to be a useful complement to antibody technology in many areas. In addition, the chip mode consisting of positive and negative screening is convenient, fast and efficient. It can also be used for screening aptamers towards other proteins, and provides a good idea design and reference for other aptamer screening workers.

### BRIEF DESCRIPTION OF DRAWINGS

FIG. 1, Schematics of aptamer screening based on microarray microfluidics.

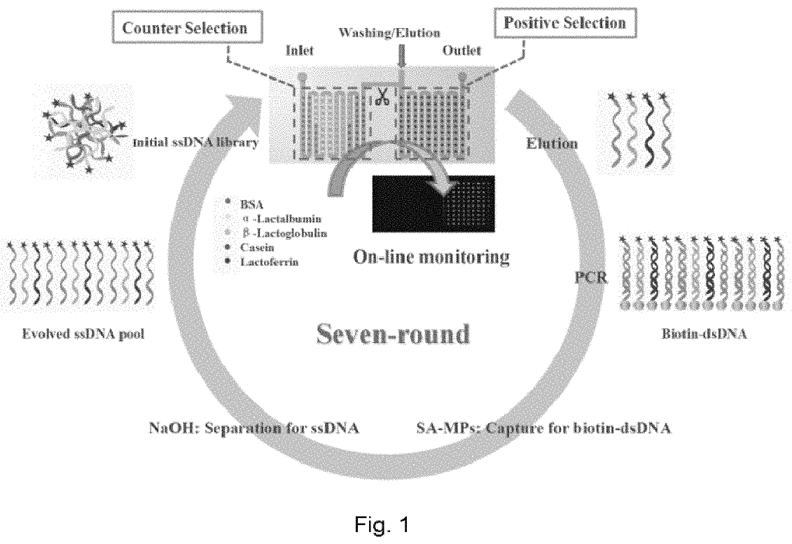

FIG. 2, Results of the first screening, (a) amount of library per round, (b) fluorescence signal intensity of the aptamer on the chip, (c) imaging image of the aptamer on the chip, (d) Image after chip cleaning.

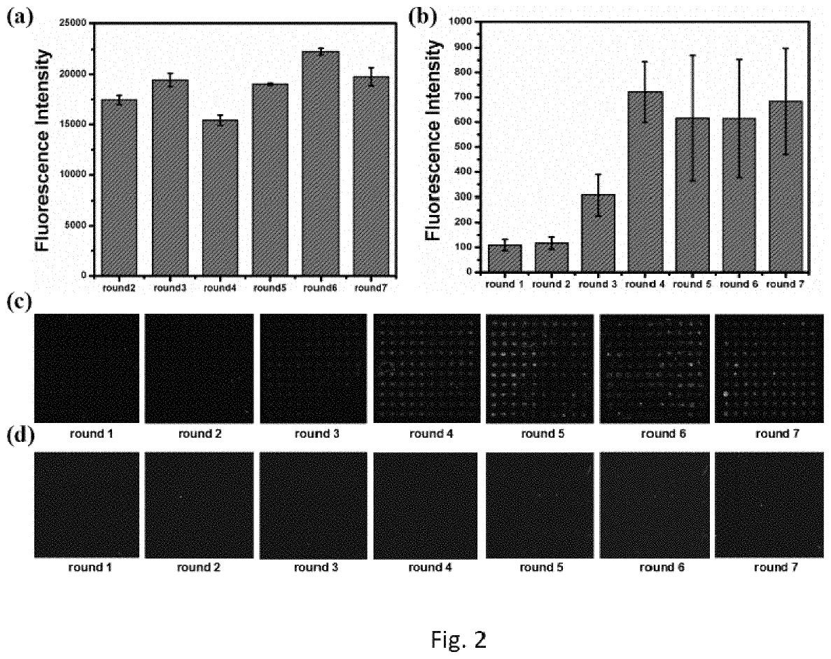

FIG. 3, Results of the second screening, (a) amount of library per round, (b) fluorescence signal intensity of the aptamer on the chip, (c) imaging image of the aptamer on the chip, (d) Image image after chip cleaning.

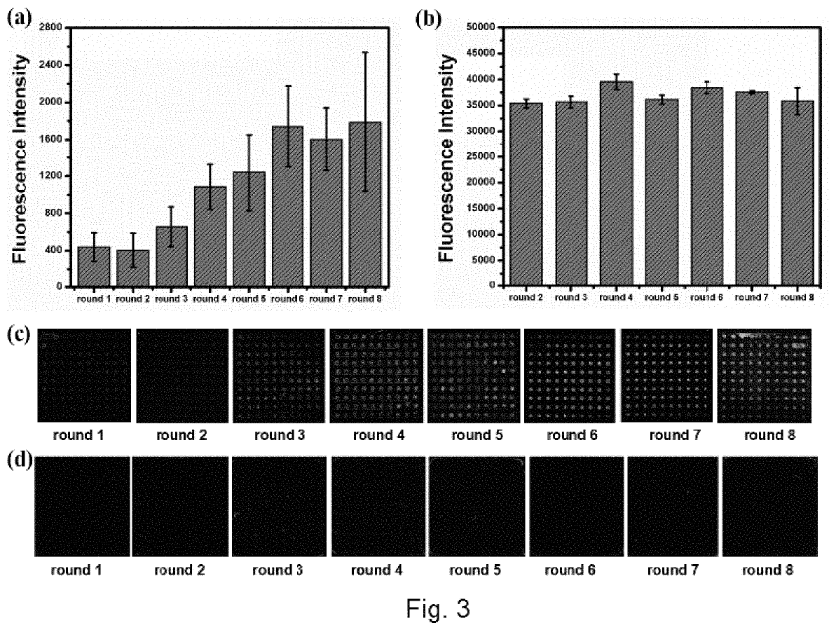

FIG. 4, Results of the third screening, (a) amount of library per round, (b) fluorescence signal intensity of the aptamer on the chip, (c) imaging image of the aptamer on the chip, (d)) Image image after chip cleaning.

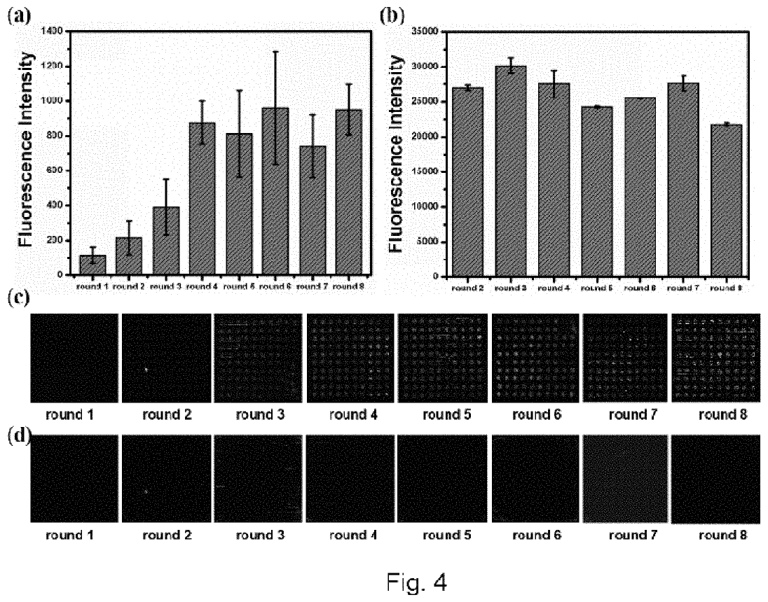

FIG. 5, A linear plot of other aptamers for lactoferrin detection by fluorescence polarization.

### DETAILED EXAMPLES

The invention can be better understood in light of the following examples. However, those skilled in the art will understand that the description of the embodiments is only intended to illustrate the invention and should not be construed as limiting the invention as described in the claims.

### Example 1: Preparation of Microarray Microfluidic Chip

Chip preparation: The microfluidic channel template was fabricated by the combination of lithography mask and chemical etching, and was reserved for the subsequent PDMS channel preparation. The PDMS pre-polymer and the curing agent are mixed at a mass ratio of 10:1. After vacuuming, it was poured onto a microfluidic channel template to obtain a PDMS microfluidic channel. A spotting instrument was used to prepare a 5 mg/mL of lactoferrin and a negative protein microarray on a glass substrate. PDMS and the spotted glass substrate are simultaneously plasma treated, and then closely adhered together as a screening chip for the next round of screening.

### Example 2: PCR Amplification

The eluted solution on the chip was divided into 6 equal volumes of 23 μL, each of which was sequentially added with 25 μL of 2×Taq polymerase, 1 μL of 20 μM TAMRA-labeled forward primer and 1 μL of 20 μM biotinylated backward primers for PCR amplification. PCR thermal cycling is as follows: 94° C. for 5 min, cycling 94° C. for 30 s, 60.5° C. for 30 s, 72° C. for 30 s with 10 rounds. The reaction was stopped at 72° C. for 5 min. The product obtained in this step was diluted 10-fold and then amplified as a template. 5 μL of the diluted PCR product, 1 μL of 2×SYBR Premix Ex Taq™ enzyme, 1 μL of 20 μM TAMRA labeled before Primer, 1 μL of 20 μM biotinylated backward primer and 18 μL of ultrapure water were mixed well. 10 μL of sample in every round was taken for fluorescence in microplate by BioTek. The round number with highest fluorescence signal was selected to amplify the remaining products.

### Example 3: Screening of Lactoferrin Aptamers

The specific steps of screening the lactoferrin aptamer include detailed processes such as chip preparation, positive and negative screening process, and PCR amplification, as described below, wherein:

Library:
(SEQ ID NO: 66)
5′-TAMRA-GACAGGCAGGACACCGTAAC-N40-CTGCTACCTCCCTCCTC
TTC-3′
TARMA modified forward primer:
(SEQ ID NO: 3)
5′-TARMA-GACAGGCAGGACACCGTAAC-3′
Biotinylated backward primer:
(SEQ ID NO: 4)
5′Biotin-GAAGAGGAGGGAGGTAGCAG-3′

(1) Chip preparation: The microfluidic channel template was fabricated by the combination of lithography mask and chemical etching, and was reserved for the subsequent PDMS channel preparation. The PDMS prepolymer and the curing agent are mixed at a mass ratio of 10:1. After vacuuming, it was poured onto a microfluidic channel template to obtain a PDMS microfluidic channel. A spotting instrument was used to prepare a 5 mg/mL of lactoferrin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 2 h at 37° C. Then 20 mg/ml of BSA and 0.01 mM of random sequence short-chain ssDNA (40 nt) were introduced and incubated at 37° C. for 1 h. Then, 150 μL of 1×PBST solution was used to clean the positive and negative screen channels.

(3) First round of screening: 0.5 nmol, 125 μL of the original library was heated at 95° C. for 5 min, and immediately frozen on ice for 10 s. Then the syringe was used to deliver the library into the positive channel at a flow rate of 2.5 μL/min. The reaction was carried out at room temperature for 50 min. Then 150 μL of 1×PBS buffer solution was passed at a flow rate of 15 μL/min to remove the unbound ssDNA sequence in the positive channel. The PDMS layer was gently torn off and Luxscan-10K/A microarray scanner is used to scan the chip. Finally, the lactoferrin-bound ssDNA sequence is eluted with DPEC water at 95° C. for 5 min, and the resulting solution is dried to a volume of 69 μL under high purity nitrogen at 50° C.

(4) PCR amplification process: the solution obtained in step (3) is divided into 3 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 600 μL of Promega beads, shake the plate for 1 h and remove the supernatant. Then 25 μL of 50 mM NaOH is added to vortex for 5 min. The double strands attached to the magnetic beads were dissociated, the supernatant was aspirated and sequentially added with 12.5 μL of 100 mM HCl, 25 μL of H₂O, 62.5 μL of 2×PBSM, and the resulting solution was used as a secondary library for the next round of screening. The content is about 40 pmol.

(6) The second round of screening: The library was pumped into the negative channel at a flow rate of 2.5 μL/min, and reacted at room temperature for 50 min. Then the pump was used to input the library into the positive channel at a flow rate of 2.5 μL/min. The reaction was carried out at room temperature for 50 min. Then 150 μL of 1×PBS buffer solution is passed at a flow rate of 15 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

### Example 4: Screening of Lactoferrin Aptamers

The specific steps of screening the lactoferrin aptamer include detailed processes such as chip preparation, positive and negative screening process, and micro PCR amplification, as described below, wherein:

Library:
(SEQ ID NO: 66)
5′-TAMRA-GACAGGCAGGACACCGTAAC-N40-CTGCTACCTCCCTCCTC
TTC-3′
TARMA modified forward primer:
(SEQ ID NO: 3)
5′-TARMA-GACAGGCAGGACACCGTAAC-3′
Biotinylated backward primer:
(SEQ ID NO: 4)
5′Biotin-GAAGAGGAGGGAGGTAGCAG-3′

(1) Chip preparation: The microfluidic channel template was fabricated by the combination of lithography mask and chemical etching, and was reserved for the subsequent PDMS channel preparation. The PDMS prepolymer and the curing agent are mixed at a mass ratio of 10:1. After vacuuming, it was poured onto a microfluidic channel template to obtain a PDMS microfluidic channel. A spotting instrument was used to prepare a 2.5 mg/mL of lactoferrin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 3 h at 37° C. Then 20 mg/ml of BSA and 0.01 mM of random sequence short-chain ssDNA (40 nt) were introduced and incubated at 37° C. for 2 h. Then, 150 μL of 1×PBST solution was used to clean the positive and negative screen channels.

(3) First round of screening: 1 nmol, 125 μL of the original library was heated at 95° C. for 5 min, and immediately frozen on ice for 10 s. Then the syringe was used to deliver the library into the positive channel at a flow rate of 2.5 μL/min. The reaction was carried out at room temperature for 50 min. Then 150 μL of 1×PBS buffer solution was passed at a flow rate of 15 μL/min to remove the unbound ssDNA sequence in the positive channel. The PDMS layer was gently torn off and Luxscan-10K/A microarray scanner is used to scan the chip. Finally, the lactoferrin-bound ssDNA sequence is eluted with DPEC water at 95° C. for 5 min, and the resulting solution is dried to a volume of 92 μL under high purity nitrogen at 50° C.

(4) PCR amplification process: the solution obtained in step (3) is divided into 4 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 800 μL of Promega beads, shake the plate for 1 h and remove the supernatant. Then 25 μL of 1 M NaOH is added to vortex for 5 min. The double strands attached to the magnetic beads were dissociated, the supernatant was aspirated and sequentially added with 12.5 μL of 2 M HCl, 25 μL of H₂O, 62.5 μL of 2×PBSM, and the resulting solution was used as a secondary library for the next round of screening. The content is about 90 pmol.

(6) The second round of screening: The library was pumped into the negative channel at a flow rate of 2.5 μL/min, and reacted at room temperature for 50 min. Then the pump was used to input the library into the positive channel at a flow rate of 2.5 μL/min. The reaction was carried out at room temperature for 50 min. Then 150 μL of 1×PBS buffer solution is passed at a flow rate of 15 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

### Example 5: Screening of Lactoferrin Aptamers

The specific steps of screening the lactoferrin aptamer include detailed processes such as chip preparation, positive and negative screening process, and micro PCR amplification, as described below, wherein:

Library:
(SEQ ID NO: 66)
5′-TAMRA-GACAGGCAGGACACCGTAAC-N40-CTGCTACCTCCCTCCTC
TTC-3′
TARMA modified forward primer:
(SEQ ID NO: 3)
5′-TARMA-GACAGGCAGGACACCGTAAC-3′
Biotinylated backward primer:
(SEQ ID NO: 4)
5′Biotin-GAAGAGGAGGGAGGTAGCAG-3′

(1) Chip preparation: The microfluidic channel template was fabricated by the combination of lithography mask and chemical etching, and was reserved for the subsequent PDMS channel preparation. The PDMS pre-polymer and the curing agent are mixed at a mass ratio of 10:1. After vacuuming, it was poured onto a microfluidic channel template to obtain a PDMS microfluidic channel. A spotting instrument was used to prepare a 5 mg/mL of lactoferrin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 2 h at 37° C. Then 20 mg/ml of BSA and 0.01 mM of random sequence short-chain ssDNA (40 nt) were introduced and incubated at 37° C. for 1 h. Then, 150 μL of 1×PBST solution was used to clean the positive and negative screen channels.

(3) First round of screening: 1 nmol, 125 μL of the original library was heated at 95° C. for 5 min, and immediately frozen on ice for 10 s. Then the syringe was used to deliver the library into the positive channel at a flow rate of 5 μL/min. The reaction was carried out at room temperature for 50 min. Then 150 μL of 1×PBS buffer solution was passed at a flow rate of 30 μL/min to remove the unbound ssDNA sequence in the positive channel. The PDMS layer was gently torn off and Luxscan˜10K/A microarray scanner is used to scan the chip. Finally, the lactoferrin-bound ssDNA sequence is eluted with DPEC water at 95° C. for 5 min, and the resulting solution is dried to a volume of 92 μL under high purity nitrogen at 50° C.

(4) PCR amplification process: the solution obtained in step (3) is divided into 4 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 800 μL of Promega beads, shake the plate for 1 h and remove the supernatant. Then 25 μL of 50 mM NaOH is added to vortex for 5 min. The double strands attached to the magnetic beads were dissociated, the supernatant was aspirated and sequentially added with 12.5 μL of 100 mM HCl, 25 μL of H₂O, 62.5 μL of 2×PBSM, and the resulting solution was used as a secondary library for the next round of screening. The content is about 60 pmol.

(6) The second round of screening: The library was pumped into the negative channel at a flow rate of 5 μL/min, and reacted at room temperature for 50 min. Then the pump was used to input the library into the positive channel at a flow rate of 5 μL/min. The reaction was carried out at room temperature for 50 min. Then 150 μL of 1×PBS buffer solution is passed at a flow rate of 30 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

### Example 6: Screening of Lactoferrin Aptamers

The specific steps of screening the lactoferrin aptamer include detailed processes such as chip preparation, positive and negative screening process, and micro PCR amplification, as described below, wherein:

Library:
(SEQ ID NO: 66)
5′-TAMRA-GACAGGCAGGACACCGTAAC-N40-CTGCTACCTCCCTCCTC
TTC-3′
TARMA modified forward primer:
(SEQ ID NO: 3)
5′-TARMA-GACAGGCAGGACACCGTAAC-3′
Biotinylated backward primer:
(SEQ ID NO: 4)
5′Biotin-GAAGAGGAGGGAGGTAGCAG-3′

(1) Chip preparation: The microfluidic channel template was fabricated by the combination of lithography mask and chemical etching, and was reserved for the subsequent PDMS channel preparation. The PDMS prepolymer and the curing agent are mixed at a mass ratio of 10:1. After vacuuming, it was poured onto a microfluidic channel template to obtain a PDMS microfluidic channel. A spotting instrument was used to prepare a 5 mg/mL of lactoferrin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 2 h at 37° C. Then 20 mg/ml of BSA and 0.01 mM of random sequence short-chain ssDNA (40 nt) were introduced and incubated at 37° C. for 1 h. Then, 150 μL of 1×PBST solution was used to clean the positive and negative screen channels.

(3) First round of screening: 1 nmol, 125 μL of the original library was heated at 95° C. for 5 min, and immediately frozen on ice for 10 s. Then the syringe was used to deliver the library into the positive channel at a flow rate of 2.5 μL/min. The reaction was carried out at room temperature for 50 min. Then 150 μL of 1×PBS buffer solution was passed at a flow rate of 15 μL/min to remove the unbound ssDNA sequence in the positive channel. The PDMS layer was gently torn off and Luxscan-10K/A microarray scanner is used to scan the chip. Finally, the lactoferrin-bound ssDNA sequence is eluted with DPEC water at 95° C. for 5 min, and the resulting solution is dried to a volume of 92 μL under high purity nitrogen at 50° C.

(4) PCR amplification process: the solution obtained in step (3) is divided into 4 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 800 μL of Promega beads, shake the plate for 1 h and remove the supernatant. Then 25 μL of 50 mM NaOH is added to vortex for 5 min. The double strands attached to the magnetic beads were dissociated, the supernatant was aspirated and sequentially added with 12.5 μL of 100 mM HCl, 25 μL of H₂O, 62.5 μL of 2×PBSM, and the resulting solution was used as a secondary library for the next round of screening. The content is about 60 pmol.

(6) The second round of screening: The library was pumped into the negative channel at a flow rate of 5 μL/min, and reacted at room temperature for 50 min. Then the pump was used to input the library into the positive channel at a flow rate of 5 μL/min. The reaction was carried out at room temperature for 50 min. Then 150 μL of 1×PBS buffer solution is passed at a flow rate of 30 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

The sequence of the resulting test aptamer is shown in SEQ ID NO. 5 to SEQ ID NO.65.

### Example 7: Detection of Lactoferrin Standard Samples by Fluorescence Polarization

(1) 100 μL of 25 μg/mL lactoferrin standard sample is mixed with 10 μL of 250 nM FITC (fluorescein isothiocyanate)-labeled aptamer N2 (base sequence:

(SEQ ID NO: 06)
AGGCAGGACACCGTAACCGGTGCATCTATGGCTACTAGCTTTTCCTGCC
T).

(2) The mixture of step (1) is placed in a 96-well plate at 37° C. for 15 min, and directly scanned with a BioTeK microplate reader. The excitation wavelength is 480 nm and the emission wavelength is 528 nm.

### Example 8: Method for Detecting Lactoferrin Content

(1) 25-100 fold diluted milk sample is mixed with 10 μL of 250 nM FITC solution (fluorescein isothiocyanate)-labeled aptamer N2 (base sequence:

(SEQ ID NO: 06)
AGGCAGGACACCGTAACCGGTGCATCTATGGCTACTAGCTTTTCCTGCC
T).

(2) The mixture of the step (1) is placed in a 96-well plate at 37° C. for 15 min, and directly scanned with a BioTeK microplate reader with an excitation wavelength of 480 nm and an emission wavelength of 528 nm.

(3) Compare the standard curve to obtain the concentration of lactoferrin in the milk sample.

### Example 9: Method for Detecting Lactoferrin Content

(1) 25-100 fold diluted milk sample is mixed with 10 μL of 250 nM FITC solution (fluorescein isothiocyanate)-labeled aptamer N6 (base sequence: gcaggacacc gtaactcggg caaagctctg aataatgttc aaccaatatt ctgtcctgc) (SEQ ID NO: 10), and mix well.

(2) The mixture of the step (1) is placed in a 96-well plate at 37° C. for 15 min, and directly scanned with a BioTeK microplate reader with an excitation wavelength of 480 nm and an emission wavelength of 528 nm.

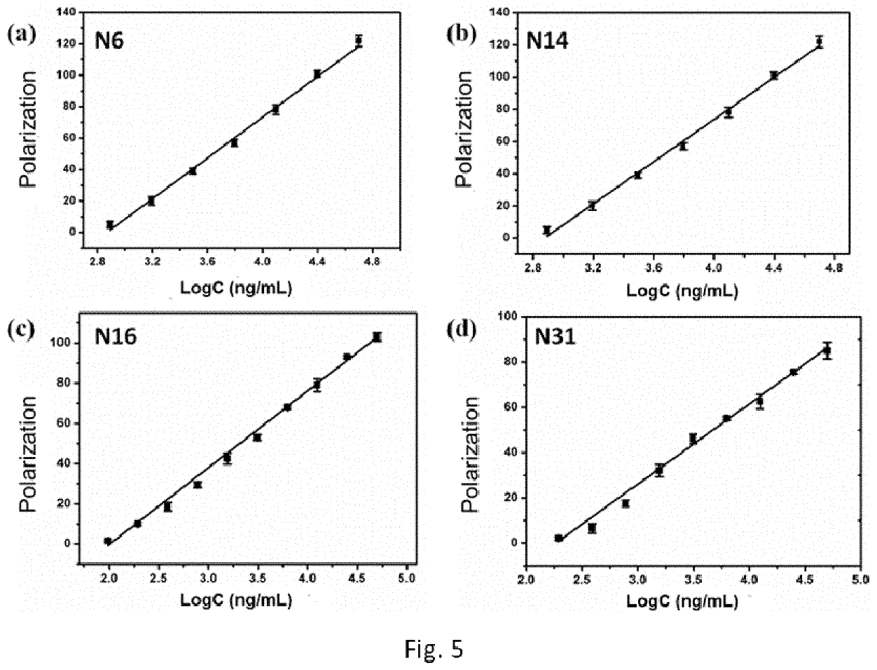

(3) Compare the standard curve to obtain the concentration of lactoferrin in the milk sample, as shown in FIG. 5(a).

### Example 10: Method for Detecting Lactoferrin Content

(1) 25-100 fold diluted milk sample is mixed with 10 μL of 250 nM FITC solution (fluorescein isothiocyanate)-labeled aptamer N14 (base sequence: gcaggacacc gtaacactgc tttatccccg tcggcttggc tcttcgacag tgtggctgc) (SEQ ID NO: 18), and mix well.

(2) The mixture of the step (1) is placed in a 96-well plate at 37° C. for 15 min, and directly scanned with a BioTeK microplate reader with an excitation wavelength of 480 nm and an emission wavelength of 528 nm.

(3) Compare the standard curve to obtain the concentration of lactoferrin in the milk sample, as shown in FIG. 5(b).

### Example 11: Method for Detecting Lactoferrin Content

(1) 25-100 fold diluted milk sample is mixed with 10 μL of 250 nM FITC solution (fluorescein isothiocyanate)-labeled aptamer N16 (base sequence: ggcaggacac cgtaacccct agttcctggt gcatttatgg caaagctttt cctgcc) (SEQ ID NO: 20), and mix well.

(2) The mixture of the step (1) is placed in a 96-well plate at 37° C. for 15 min, and directly scanned with a BioTeK microplate reader with an excitation wavelength of 480 nm and an emission wavelength of 528 nm.

(3) Compare the standard curve to obtain the concentration of lactoferrin in the milk sample, as shown in FIG. 5(c).

### Example 12: Method for Detecting Lactoferrin Content

(1) 25-100 fold diluted milk sample is mixed with 10 μL of 250 nM FITC solution (fluorescein isothiocyanate)-labeled aptamer N31 (ggcaggacac cgtaaccagt ataggtgcat ttttggcgca agctcttcct gccctg) (SEQ ID NO: 35), and mix well.

(2) The mixture of the step (1) is placed in a 96-well plate at 37° C. for 15 min, and directly scanned with a BioTeK microplate reader with an excitation wavelength of 480 nm and an emission wavelength of 528 nm.

(3) Compare the standard curve to obtain the concentration of lactoferrin in the milk sample, as shown in FIG. 5(d).

### Example 13: Screening of α-Lactalbumin Aptamers

The specific steps of the α-lactalbumin aptamer screening include detailed processes such as chip preparation, positive and negative screening process, and PCR amplification, as described below, wherein:

Library:
(SEQ ID NO: 66)
5′-TAMRA-GACAGGCAGGACACCGTAAC-N40-CTGCTACCTCCCTCCTC
TTC-3′
TARMA modified forward primer:
(SEQ ID NO: 3)
5′-TARMA-GACAGGCAGGACACCGTAAC-3′
Biotinylated backward primer:
(SEQ ID NO: 4)
5′Biotin-GAAGAGGAGGGAGGTAGCAG-3′

(1) Chip preparation: The microfluidic channel template was fabricated by the combination of lithography mask and chemical etching, and was reserved for the subsequent PDMS channel preparation. The PDMS prepolymer and the curing agent are mixed at a mass ratio of 10:1. After vacuuming, it was poured onto a microfluidic channel template to obtain a PDMS microfluidic channel. A spotting instrument was used to prepare a 5 mg/mL of lactoferrin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 2 h at 37° C. Then 20 mg/ml of BSA and 0.01 mM of random sequence short-chain ssDNA (40 nt) were introduced and incubated at 37° C. for 1 h. Then, 150 μL of 1×PBST solution was used to clean the positive and negative screen channels.

(3) First round of screening: 0.5 nmol, 125 μL of the original library was heated at 95° C. for 5 min, and immediately frozen on ice for 10 s. Then the syringe was used to deliver the library into the positive channel at a flow rate of 2.5 μL/min. The reaction was carried out at room temperature for 50 min. Then 150 μL of 1×PBS buffer solution was passed at a flow rate of 15 μL/min to remove the unbound ssDNA sequence in the positive channel. The PDMS layer was gently torn off and Luxscan-10K/A microarray scanner is used to scan the chip. Finally, the α-lactalbumin-bound ssDNA sequence is eluted with DPEC water at 95° C. for 5 min, and the resulting solution is dried to a volume of 92 μL under high purity nitrogen at 50° C.

(4) PCR amplification process: the solution obtained in step (3) is divided into 4 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 800 μL of Promega beads, shake the plate for 1 h and remove the supernatant. Then 25 μL of 50 mM NaOH is added to vortex for 5 min. The double strands attached to the magnetic beads were dissociated, the supernatant was aspirated and sequentially added with 12.5 μL of 100 mM HCl, 25 μL of H₂O, 62.5 μL of 2×PBSM, and the resulting solution was used as a secondary library for the next round of screening. The content is about 40 pmol.

(6) The second round of screening: The library was pumped into the negative channel at a flow rate of 2.5 μL/min, and reacted at room temperature for 50 min. Then the pump was used to input the library into the positive channel at a flow rate of 2.5 μL/min. The reaction was carried out at room temperature for 50 min. Then 150 μL of 1×PBS buffer solution is passed at a flow rate of 15 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

### Example 14: Screening of β-Lactoglobulin Aptamers

The specific steps of the β-lactoglobulin aptamer screening include detailed processes such as chip preparation, positive and negative screening process, and PCR amplification, as described below, wherein:

Library:
(SEQ ID NO: 66)
5'-TAMRA-GACAGGCAGGACACCGTAAC-N40-CTGCTACCTCCCTCCTC
TTC-3'
TARMA modified forward primer:
(SEQ ID NO: 3)
5'-TARMA-GACAGGCAGGACACCGTAAC-3'
Biotinylated backward primer:
(SEQ ID NO: 4)
5'Biotin-GAAGAGGAGGGAGGTAGCAG-3'

(1) Chip preparation: The microfluidic channel template was fabricated by the combination of lithography mask and chemical etching, and was reserved for the subsequent PDMS channel preparation. The PDMS prepolymer and the curing agent are mixed at a mass ratio of 10:1. After vacuuming, it was poured onto a microfluidic channel template to obtain a PDMS microfluidic channel. A spotting instrument was used to prepare a 5 mg/mL of β-lactoglobulin and a negative protein microarray on a glass substrate. PDMS and the spotted glass substrate are simultaneously plasma treated, and then closely adhered together as a screening chip for the next round of screening.

(2) Screening preparation: The screening chip obtained in step (1) was placed in a constant temperature water bath for incubation for 2 h at 37° C. Then 20 mg/ml of BSA and 0.01 mM of random sequence short-chain ssDNA (40 nt) were introduced and incubated at 37° C. for 1 h. Then, 150 μL of 1×PBST solution was used to clean the positive and negative screen channels.

(3) First round of screening: 0.5 nmol, 125 μL of the original library was heated at 95° C. for 5 min, and immediately frozen on ice for 10 s. Then the syringe was used to deliver the library into the positive channel at a flow rate of 2.5 μL/min. The reaction was carried out at room temperature for 50 min. Then 150 μL of 1×PBS buffer solution was passed at a flow rate of 15 μL/min to remove the unbound ssDNA sequence in the positive channel. The PDMS layer was gently torn off and Luxscan-10K/A microarray scanner is used to scan the chip. Finally, the β-lactoglobulin-bound ssDNA sequence is eluted with DPEC water at 95° C. for 5 min, and the resulting solution is dried to a volume of 92 μL under high purity nitrogen at 50° C.

(4) PCR amplification process: the solution obtained in step (3) is divided into 4 parts of the same solution with a volume of 23 μL. Each step is added with 25 μL of 2×Taq polymerase, 1 μL of 20 μM TAMRA labeled forward primers and 1 μL of 20 μM biotinylated backward primers and the mixture were PCR-amplified. PCR thermal cycling is as follows: 94° C. for 5 min, cycling 94° C. for 30 s, 60.5° C. for 30 s, 72° C. 30 s for 10 rounds and terminated at 72° C. for 5 min. The product obtained in this step is diluted 10 times and then amplified as a template: 5 μL of the diluted PCR product and 1 μL of 2×SYBR Premix Ex Taq™ enzyme, 1 μL of 20 μM TAMRA-labeled forward primer, 1 μL of 20 μM biotinylated backward primer and 18 μL DPEC water were mixed well. 10 μL of sample in every round was taken for fluorescence in microplate by BioTek. The round number with highest fluorescence signal was selected to amplify the remaining products.

(5) Separation and purification: Mix the PCR product obtained in step (4) with 800 μL of Promega beads, shake the plate for 1 h and remove the supernatant. Then 25 μL of 50 mM NaOH is added to vortex for 5 min. The double strands attached to the magnetic beads were dissociated, the supernatant was aspirated and sequentially added with 12.5 μL of 100 mM HCl, 25 μL of H₂O, 62.5 μL of 2×PBSM, and the resulting solution was used as a secondary library for the next round of screening. The content is about 40 pmol.

(6) The second round of screening: The library was pumped into the negative channel at a flow rate of 2.5 μL/min, and reacted at room temperature for 50 min. Then the pump was used to input the library into the positive channel at a flow rate of 2.5 μL/min. The reaction was carried out at room temperature for 50 min. Then 150 μL of 1×PBS buffer solution is passed at a flow rate of 15 μL/min to remove unreacted chains in the positive channel. The PDMS layer is gently torn off and Luxscan-10K/A microarray scanner is used to scan the chip. Finally, the protein-bound chain was eluted by heating with non-nuclear water at 95° C. for 5 min, and the resulting solution was dried at 60° C. to a volume of 92 μL, which was left for PCR amplification;

(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;

(8) repeating steps (5), (6) and (7) for 2^{nd}-6^{th} round screens, and repeating steps (5) and (6) for 7^{th} round screen;

(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

### Example 15: Conserved Sequence Analysis for the Lactoferrin-Bound Aptamers

The motif finder software was used to find if there a conserved sequence in the obtained lactoferrin-bound aptamers. SEQ ID: 67 (aggcaggacaccgtaaccggtgcatcyatggctwytagctyttcctg) is identified as a conserved sequence for SEQ ID: 6, SEQ ID: 22 and SEQ ID: 41.

What is claimed is:

1. A method for screening lactoferrin-bound aptamers by using a microarray microfluidic chip, the microarray microfluidic chip is prepared as follows:
(1) mixing a PDMS pre-polymer and a curing agent at a mass ratio of 5:1 to 100:1 as a mixture; transferring the mixture into a vacuum desiccator which is connected to a circulating water vacuum pump for 30 min for removing bubbles which yields a non-bubble mixture; and pouring the non-bubble mixture on a microfluidic channel template which yields a poly-di-methyl-siloxane (PDMS) microfluidic channel; preparing a lactoferrin microarray with concentration of 0.1-10 mg/mL on a glass substrate and a microarray without lactoferrin on the glass substrate; treating the glass substrate and the PDMS microfluidic channel simultaneously with a plasma which yields a positive microarray microfluidic chip with lactoferrin and a negative microarray microfluidic chip without lactoferrin;
(2) incubating the positive and negative microarray microfluidic chips for 1-12 hours at 25-40° C.; putting the positive and negative microarray microfluidic chips into 5-20 mg/ml of BSA and 0.01-0.5 mM of 40 nucleotide random sequence ssDNA at 25-40° C. for 1-3 hours, and washing the positive and negative microfluidic chips with 150 μL of 1×PBST solution;
(3) heating 0.1-10 nmol, 125 μL of an original library for 3-15 min at 95° C., and immediately freezing on ice for 10 sec-5 min; transferring the library into a positive microfluidic channel by a syringe pump at a flow rate of 1-5 μL/min, reacting for 30-120 min at 25-40° C.; injecting 150 μL of 1×PBS buffer at a flow rate of 5-30 μL/min in the positive microfluidic channel; peeling a PDMS layer and scanning the microarray microfluidic chip by a microarray scanner; eluting lactoferrin-bound ssDNA by heating with DPEC water for 3-10 min at 95° C., and drying resulting solution up to a volume of 25-250 μL; wherein the original library consists of 40 random bases in middle of the DNA, an oligonucleotide having sequence shown in SEQ ID NO. 1 at 5′ end, and an oligonucleotide having sequence shown in SEQ ID NO. 2 at 3′ end;
(4) dividing the solution obtained in step (3) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;
(5) mixing PCR products in step (4) with 800 μL-5 mL of streptavidin magnetic beads, removing supernatant after rapid shaking for 0.5-2 h; adding 25 μL of 0.01-0.1 M NaOH solution into the supernatant and mixing the supernatant for 3-15 min with a vortex mixer; adding 12.5 μL of 0.1M HCl, 25 μL of DPEC water and 62.5 μL of 2×PBSM buffer solution into the supernatant which is used as a secondary library for next round of screening with concentration of 40 pmol-100 pmol;
(6) transferring the secondary library into a negative microfluidic channel at 1-5 μL/min at 25-40° C., transferring into the positive microfluidic channel; washing the positive microfluidic channel with 150 μL of 1×PBS buffer solution at 5-30 μL/min flow rate; peeling the PDMS layer; eluting lactoferrin-bound ssDNA on the microarray microfluidic chip with DPEC water by heating for 3-10 min at 95° C., and drying solution up to 20-250 μL at 60° C. for PCR amplification products;
(7) dividing the solution obtained in step (6) into 3 to 10 parts with a volume of 23 μL, adding 25 μL of 2×Taq polymerase, 1 μL of 20 μM TAMRA labeled forward primer and 1 μL of 20 μM biotinylated backward primer into each part for PCR amplification; diluting PCR product by 5 to 40 times as a template; mixing 5 μL of the diluted PCR product with 1 μL of Taq polymerase, 1 μL of 20 μM TAMRA-labeled forward primer, 1 μL of 20 μM biotinylated backward primer and 18 μL of water for PCR amplification; taking 10 μL of PCR product into a microplate for fluorescence assay; selecting PCR products with fluorescence signal for amplifying;
(8) repeating steps (5), (6) and (7) for 2^{nd}-6^{th} round screens, and repeating steps (5) and (6) for 7^{th} round screen;
(9) sequencing 5^{th}, 6^{th}, 7^{th} round screens of the PCR amplification products in the step (8); repeating screening and randomly selecting samples for sequencing; sequences with a repeated sequence greater than 2 are obtained, and the sequences being capable of generating secondary structures are the lactoferrin-bound aptamers.

2. The method according to claim 1, wherein in step (4), the forward primer has a nucleotide sequence shown in SEQ ID NO.3, the backward primer has a nucleotide sequence shown in SEQ ID NO.4.

3. A lactoferrin-bound aptamer, wherein the lactoferrin-bound aptamer is a polynucleotide having nucleotide sequence shown as SEQ ID No: 67.

4. A method for detecting lactoferrin content comprising a step for determining amount of an aptamer that interacted with the lactoferrin, wherein the aptamer is a polynucleotide having nucleotide sequence shown as SEQ ID NO: 67.


SEQUENCE LISTING (67 sequences)

SEQ ID NO: 1 - DNA, 20 bp, Artificial Sequence
  Nucleotide sequence at the 5′ end of a random base which is synthesized
gacaggcaggacaccgtaac

SEQ ID NO: 2 - DNA, 20 bp, Artificial Sequence
  Nucleotide sequence at the 3′ end of a random base which is synthesized.
ctgctacctccctcctcttc

SEQ ID NO: 3 - DNA, 20 bp, Artificial Sequence
  Forward primer that is synthesized
gacaggcaggacaccgtaac

SEQ ID NO: 4 - DNA, 20 bp, Artificial Sequence
  Backward primer that is synthesized.
gaagaggagggaggtagcag

SEQ ID NO: 5 - DNA, 59 bp, Artificial Sequence
  AptamerN1 that is synthesized
gcaggacaccgtaaccctaacacgtacggggcatttatggcatagctcttcctccctgc

SEQ ID NO: 6 - DNA, 50 bp, Artificial Sequence
  AptamerN2 that is synthesized
aggcaggacaccgtaaccggtgcatctatggctactagcttttcctgcct

SEQ ID NO: 7 - DNA, 59 bp, Artificial Sequence
  AptamerN3 that is synthesized
gcaggacaccgtaacgggcaatcttgctcttattttcacacttgataaatatgtcctgc

SEQ ID NO: 8 - DNA, 59 bp, Artificial Sequence
  AptamerN4 that is synthesized
gcaggacaccgtaacgggcgaacgctctaaataaatggcatcaatttcttttgccctgc

SEQ ID NO: 9 - DNA, 59 bp, Artificial Sequence
  AptamerN5 that is synthesized
gcaggacaccgtaacgggcttatgctcttaaaaatcctgagcgactttttatgtactgc

SEQ ID NO: 10 - DNA, 59 bp, Artificial Sequence
  AptamerN6 that is synthesized
gcaggacaccgtaactcgggcaaagctctgaataatgttcaaccaatattctgtcctgc

SEQ ID NO: 11 - DNA, 59 bp, Artificial Sequence
  AptamerN7 that is synthesized
gcaggacaccgtaacaatactcctgttaccgtgcatctatggccattggcttttcctgc

SEQ ID NO: 12 - DNA, 59 bp, Artificial Sequence
  AptamerN8 that is synthesized
gcaggacaccgtaacacgggctctgcttttccaataatgacaagtattggatggcctgc

SEQ ID NO: 13 - DNA, 59 bp, Artificial Sequence
  AptamerN9 that is synthesized
gcaggacaccgtaacactctcaaccatgacggtgcaatattggcgctagctcttcctgc

SEQ ID NO: 14 - DNA, 59 bp, Artificial Sequence
  AptamerN10 that is synthesized
gcaggacaccgtaacgtattaactagtaccagtgcatctatggctattgcttttcctgc

SEQ ID NO: 15 - DNA, 59 bp, Artificial Sequence
  AptamerN11 that is synthesized
gcaggacaccgtaacgttcgtaacaacacggcatctatggctttagctcttgtacctgc

SEQ ID NO: 16 - DNA, 52 bp, Artificial Sequence
  AptamerN12 that is synthesized
aggcaggacaccgtaacaccggtgcgtcccacggctccagctcttcctgcct

SEQ ID NO: 17 - DNA, 59 bp, Artificial Sequence
  AptamerN13 that is synthesized
gcaggacaccgtaacacgggctgatgctctctttattttacctaaataaagtgtcctgc

SEQ ID NO: 18 - DNA, 59 bp, Artificial Sequence
  AptamerN14 that is synthesized
gcaggacaccgtaacactgctttatccccgtcggcttggctcttcgacagtgtggctgc

SEQ ID NO: 19 - DNA, 40 bp, Artificial Sequence
  AptamerN15 that is synthesized
agcagacgggagacctttagagttgtaacttgagtctgct

SEQ ID NO: 20 - DNA, 56 bp, Artificial Sequence
  AptamerN16 that is synthesized
ggcaggacaccgtaacccctagttcctggtgcatttatggcaaagcttttcctgcc

SEQ ID NO: 21 - DNA, 61 bp, Artificial Sequence
  AptamerN17 that is synthesized
caggcaggacaccgtaaccgggcatttgctctcaatttagtctcaaattgtggcctgcctg

SEQ ID NO: 22 - DNA, 50 bp, Artificial Sequence
  AptamerN18 that is synthesized
aggcaggacaccgtaaccggtgcatctatggctactagctcttcctgcct

SEQ ID NO: 23 - DNA, 59 bp, Artificial Sequence
  AptamerN19 that is synthesized
gcaggacaccgtaacgggcgaacgctctttattttggaacccacaaaataatgttctgc

SEQ ID NO: 24 - DNA, 59 bp, Artificial Sequence
  AptamerN20 that is synthesized
gcaggacaccgtaacgggcggagctcttccatttcagatttgtaaatggatgtcactgc

SEQ ID NO: 25 - DNA, 59 bp, Artificial Sequence
  AptamerN21 that is synthesized
gcaggacaccgtaacgggctatgctctaaattcttcctactgacgcaattttggactgc

SEQ ID NO: 26 - DNA, 59 bp, Artificial Sequence
  AptamerN22 that is synthesized
gcaggacaccgtaacgggtgcacactcttatttttacacgagccaaaaatatgtcctgc

SEQ ID NO: 27 - DNA, 61 bp, Artificial Sequence
  AptamerN23 that is synthesized
caggcaggacaccgtaacggtgcaattattggtgaacactcttccttagcctgctactctg

SEQ ID NO: 28 - DNA, 59 bp, Artificial Sequence
  AptamerN24 that is synthesized
gcaggacaccgtaactcatcccaagctccggtgccatctatgggcttcgcttttcctgc

SEQ ID NO: 29 - DNA, 52 bp, Artificial Sequence
  AptamerN25 that is synthesized
aggcaggacaccgtaactgaacggtcttggctactgcacatttttcctgcct

SEQ ID NO: 30 - DNA, 59 bp, Artificial Sequence
  AptamerN26 that is synthesized
gcaggacaccgtaacaacaacttcgtatccggtgcatttatggcgaatgcttttcctgc

SEQ ID NO: 31 - DNA, 59 bp, Artificial Sequence
  AptamerN27 that is synthesized
gcaggacaccgtaacactaccttctttcggtgcgatccattcggctttgcttttcctgc

SEQ ID NO: 32 - DNA, 59 bp, Artificial Sequence
  AptamerN28 that is synthesized
gcaggacaccgtaacactgctttatccccgtcggcttggctcttcgacagtgttgctgc

SEQ ID NO: 33 - DNA, 59 bp, Artificial Sequence
  AptamerN29 that is synthesized
gcaggacaccgtaacactgctttatccccgtcggcttgtctcttcgacagtgtggctgc

SEQ ID NO: 34 - DNA, 40 bp, Artificial Sequence
  AptamerN30 that is synthesized
agcagacgggagacttttagagttgtaacttgagtctgct

SEQ ID NO: 35 - DNA, 56 bp, Artificial Sequence
  AptamerN31 that is synthesized
ggcaggacaccgtaaccagtataggtgcatttttggcgcaagctcttcctgccctg

SEQ ID NO: 36 - DNA, 56 bp, Artificial Sequence
  AptamerN32 that is synthesized
ggcaggacaccgtaaccatcgacggtgccggtctcgggcattttgctcttcctgcc

SEQ ID NO: 37 - DNA, 55 bp, Artificial Sequence
  AptamerN33 that is synthesized
ggcaggacaccgtaaccccactcgaggtgcaattttggcgtgagctcttcctgcc

SEQ ID NO: 38 - DNA, 56 bp, Artificial Sequence
  AptamerN34 that is synthesized
ggcaggacaccgtaacccctagttcctggtgcatctatggcaaagctcttcctgcc

SEQ ID NO: 39 - DNA, 56 bp, Artificial Sequence
  AptamerN35 that is synthesized
ggcaggacaccgtaacccctagttcctggtgcatttatggcaaagctcttcctgcc

SEQ ID NO: 40 - DNA, 56 bp, Artificial Sequence
  Aptamer36 that is synthesized
aggcaggacaccgtaacctacaacggtgccatctatgggctttgcttttcctgcct

SEQ ID NO: 41 - DNA, 56 bp, Artificial Sequence
  Aptamer37 that is synthesized
gacaggcaggacaccgtaacggtgcatccatggcttttagctcttcctgaactgtc

SEQ ID NO: 42 - DNA, 70 bp, Artificial Sequence
  Aptamer38 that is synthesized
aggcaggacaccgtaacggtgcatctatggctttgctcttcctacctgttctacgagctgctacctccct

SEQ ID NO: 43 - DNA, 49 bp, Artificial Sequence
  Aptamer39 that is synthesized
aggcaggacaccgtaacggtgcgtgtacacggctctgcttttcctgcct

SEQ ID NO: 44 - DNA, 82 bp, Artificial Sequence
  AptamerN40 that is synthesized
tgacaggcaggacaccgtaacaacaactcgatactggtgccattttgggcgaacgcttttcctgctacctccctcctcttca

SEQ ID NO: 45 - DNA, 82 bp, Artificial Sequence
  AptamerN41 that is synthesized
tgacaggcaggacaccgtaacaaccaacctacggtgcaatatttggcggattaagctcttcctgctacctccctcctcttca

SEQ ID NO: 46 - DNA, 82 bp, Artificial Sequence
  AptamerN42 that is synthesized
tgacaggcaggacaccgtaacacacgttaacttacggcgcattttttggcttcagctcttcctgctacctccctcctcttca

SEQ ID NO: 47 - DNA, 82 bp, Artificial Sequence
  AptamerN43 that is synthesized
tgacaggcaggacaccgtaacacggtgcaaactcttggcgaatgcttttcctgacccgctactgctacctccctcctcttca

SEQ ID NO: 48 - DNA, 82 bp, Artificial Sequence
  AptamerN44 that is synthesized
tgacaggcaggacaccgtaacatacgattctctcgcacattgtggctccgcttttatgttactgctacctccctcctcttca

SEQ ID NO: 49 - DNA, 82 bp, Artificial Sequence
  AptamerN45 that is synthesized
tgacaggcaggacaccgtaaccatacttcggtgcatcccatggcgaattcgctcttccttcctgctacctccctcctcttca

SEQ ID NO: 50 - DNA, 82 bp, Artificial Sequence
  AptamerN46 that is synthesized
tgacaggcaggacaccgtaaccgggcggagctcttattacatcaactcgctgtaatatggcctgctacctccctcctcttca

SEQ ID NO: 51 - DNA, 82 bp, Artificial Sequence
  AptamerN47 that is synthesized
tgacaggcaggacaccgtaacgggcaatcttgctcttattttcacacctgataaatatgtcctgctacctccctcctcttca

SEQ ID NO: 52 - DNA, 82 bp, Artificial Sequence
  AptamerN48 that is synthesized
tgacaggcaggacaccgtaacgggcttagctctaaatcaaatcctccttattgattttgtcctgctacctccctcctcttca

SEQ ID NO: 53 - DNA, 82 bp, Artificial Sequence
  AptamerN49 that is synthesized
tgacaggcaggacaccgtaacgggtaacactctacttataccttgctataagttgtccgcactgctacctccctcctcttca

SEQ ID NO: 54 - DNA, 82 bp, Artificial Sequence
  AptamerN50 that is synthesized
tgacaggcaggacaccgtaacgggtttaactctcaaattagctctgcactacatttgtgccctgctacctccctcctcttca

SEQ ID NO: 55 - DNA, 82 bp, Artificial Sequence
  AptamerN51 that is synthesized
tgacaggcaggacaccgtaacgtaactacggacggcatctatggctatgctcttgtccctgctgctacctccctcctcttca

SEQ ID NO: 56 - DNA, 82 bp, Artificial Sequence
  AptamerN52 that is synthesized
tgacaggcaggacaccgtaactacctcttccatatcggtgcaacatttggcttagctcttcctgctacctccctcctcttca

SEQ ID NO: 57 - DNA, 82 bp, Artificial Sequence
  AptamerN53 that is synthesized
tgacaggcaggacaccgtaacttctgataatcggtgcatctatggcgtagctcttcctcacctgctacctccctcctcttca

SEQ ID NO: 58 - DNA, 82 bp, Artificial Sequence
  AptamerN54 that is synthesized
tgacaggcaggacaccgtaacacggggcaattttggtgaagctcttcctgcccagcctactctgctacctccctcctcttca

SEQ ID NO: 59 - DNA, 82 bp, Artificial Sequence
  AptamerN55 that is synthesized
tgacaggcaggacaccgtaaccgggctattgctctcaaacttacactttaacgtttgggtcctgctacctccctcctcttca

SEQ ID NO: 60 - DNA, 82 bp, Artificial Sequence
  AptamerN56 that is synthesized
tgacaggcaggacaccgtaacgcgggcgaagctcttaatacctctctattatgtcctgttcctgctacctccctccccttca

SEQ ID NO: 61 - DNA, 82 bp, Artificial Sequence
  AptamerN57 that is synthesized
tgacaggcaggacaccgtaacgggcgaatgctctctttgttttatatataacaaagtgtctctgctacctccctcctcttca

SEQ ID NO: 62 - DNA, 82 bp, Artificial Sequence
  AptamerN58 that is synthesized
tgacaggcaggacaccgtaacgggctctgctctaaatttcgcccttgcacgaaattttgtcctgctacctcccccctcttca

SEQ ID NO: 63 - DNA, 82 bp, Artificial Sequence
  AptamerN59 that is synthesized
tgacaggcaggacaccgtaacgggcttatgctcttaaaaatcctgagcgactttttatgtactgctacctccctcctcttca

SEQ ID NO: 64 - DNA, 82 bp, Artificial Sequence
  AptamerN60 that is synthesized
tgacaggcaggacaccgtaacggtgcggtcatatccggctttgcttttcctgccttctctcctgctacctccctcctcttca

SEQ ID NO: 65 - DNA, 82 bp, Artificial Sequence
  AptamerN61 that is synthesized
tgacaggcgggacaccgtaaccccaacacgtacggggcatttatggcatagctcttcctccctgctacctccctcctcttca

SEQ ID NO: 66 - DNA, 80 bp, Artificial Sequence
  Library is synthesized
  misc_feature (21)..(60): n is a, c, g, or t
gacaggcaggacaccgtaacnnnnnnnnnnnnnnnnnnnnnnnnnnnnnnnnnnnnnnnnctgctacctccctcctcttc

SEQ ID NO: 67 - DNA, 47 bp, Artificial Sequence
  It is synthesized.
aggcaggacaccgtaaccggtgcatcyatggctwytagctyttcctg